(19) United States
(12) Patent Application Publication
(10) Pub. No.: US 2006/0062823 A1
(43) Pub. Date: Mar. 23, 2006

(54) Microtubule stabilisers for treating stenosis in stents

(76) Inventor: Margaret Forney Prescott, Morris Plains, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/535,879

(22) PCT Filed: Dec. 8, 2003
(86) PCT No.: PCT/EP03/13885

§ 371 Date: May 20, 2005

Related U.S. Application Data
(60) Provisional application No. 60/431,846, filed on Dec. 9, 2002.

(51) Int. Cl. A61K 31/427; A61F 2/00

(52) U.S. Cl. 424/423; 514/365

(57) ABSTRACT

A drug delivery device or system comprising: a) a medical device, e.g. a coated stent, adapted for local application or administration in hollow tubes; and, in conjunction therewith, b) a therapeutic dosage of a MIA, e.g. epothilone B, e.g. affixed to the medical device, and corresponding use in the preparation of a medicament, and corresponding method of treatment.

The present invention relates to drug delivery systems for the prevention and treatment of proliferative diseases, particularly vascular diseases.

Many humans suffer from circulatory diseases caused by a progressive blockage of the blood vessels that perfuse the heart and other major organs. Severe blockage of blood vessels in such humans often leads to ischemic injury, hypertension, stroke or myocardial infarction. Atherosclerotic lesions which limit or obstruct coronary or periphery blood flow are the major cause of ischemic disease related morbidity and mortality including coronary heart disease and stroke. In order to stop the disease process and prevent the more advanced disease states in which the cardiac muscle or other organs are compromised, medical revascularization procedures such as percutaneous transluminal coronary angioplasty (PCTA), percutaneous transluminal angioplasty (PTA), atherectomy, bypass grafting or other types of vascular grafting procedures are used.

Re-narrowing (restenosis) of an artherosclerotic coronary artery after various revascularization procedures occurs in 10-80% of patients undergoing this treatment, depending on the procedure used and the aterial site. Besides opening an artery obstructed by atherosclerosis, revascularization also injures endothelial cells and smooth muscle cells within the vessel wall, thus initiating a thrombotic and inflammatory response. Cell derived growth factors such as platelet derived growth factor, infiltrating macrophages, leukocytes or the smooth muscle cells themselves provoke proliferative and migratory responses in the smooth muscle cells. Simultaneous with local proliferation and migration, inflammatory cells also invade the site of vascular injury and may migrate to the deeper layers of the vessel wall. Proliferation/migration usually begins within one to two days post-injury and, depending on the revascularization procedure used, continues for days and weeks.

Both cells within the atherosclerotic lesion and those within the media migrate, proliferate and/or secrete significant amounts of extracellular matrix proteins. Proliferation, migration and extracellular matrix synthesis continue until the damaged endothelial layer is repaired at which time proliferation slows within the intima. The newly formed tissue is called neointima, intimal thickening or restenotic lesion and usually results in narrowing of the vessel lumen. Further lumen narrowing may take place due to constructive remodeling, e.g. vascular remodeling, leading to further intimal thickening or hyperplasia.

remodeling, leading to further intimal thickening or hyperplasia.

Accordingly, there is a need for effective treatment and drug delivery systems for preventing and treating intimal thickening or restenosis that occurs after injury, e.g. vascular injury, including e.g. surgical injury, e.g. revascularization-induced injury, e.g. also in heart or other grafts.

Surprisingly, it has now been found that microtubule interfereing agents (MIA), optionally in conjunction with other active compounds, e.g. antiproliferative compounds, have beneficial effects when locally applied to the lesions sites.

Hence, the invention relates to a method of treating for preventing and treating intimal thickening or restenosis that occurs after injury, e.g. vascular injury, including e.g. surgical injury, e.g. revascularization-induced injury, e.g. also in heart or other grafts, comprising administering a therapeutically effective amount of an MIA to a warm-blooded animal in need thereof.

The invention particularly concerns drug delivery devices or systems comprising:
a) a medical device, e.g. a catheter-based delivery device or an intraluminal device, especially a coated stent, adapted for local application or administration in hollow tubes; and, in conjunction therewith,
b) a therapeutic dosage of a MIA, optionally together with a therapeutic dosage of one or more other active ingredients, preferably each being affixed to the medical device in a way allowing drug release;
   hereinafter briefly named “the device of the invention”.

A device of the invention preferably comprises a coated stent.

The invention also concerns the use of a MIA derivative in the preparation of a medicament for:
the prevention or treatment, e.g. systemically, preferably locally, of vascular inflammation or smooth muscle cell proliferation and migration in hollow tubes or increased inflammatory cell infiltration or increased cell proliferation or increased matrix deposition or destruction or increased remodeling following stent placement; or
the treatment of intimal thickening in vessel walls; preferably in conjunction with a medical device as defined under a) above.

MIA compounds are known and clinically used for the treatment of cancer. Such compounds include coichicine, podophyllotoxins, such as etoposide and teniposide, taxanes, such as paclitaxel and docetaxel, discodermolide compounds, which includes (+)-discodermolide and analogs and derivatives of (+)-discodermolide, vinca alkaloids, such as vinblastin, especially vinblastine sulfate, vincristine, especially vincristine sulfate, and vinorelbine, and epothilones, such as epothilones A, B, C and D, as well as analogs and derivatives thereof, for example the compounds disclosed in WO 99/02514, particularly [1S-[1R, 3R(E), 7R, 10S,11R, 12R, 16S]]-7,11-dihydroxy-8,8,10,12,16-pentamethyl-3-[1-methyl-2-(2-methyl-4-thiazolyl)ethenyl]-4-aza-17-bicyclo[14.1.0]-heptadecane-5,9-dione (example 3). Vinblastine sulfate can be administered, e.g., in the form as it is marketed, e.g. under the trademark VINBLASTIN R.P.™. Vincristine sulfate can be administered, e.g., in the form as it is marketed, e.g. under the trademark FARMISTIN™. Discodermolide can be obtained, e.g., as disclosed in U.S. Pat. Nos. 4,939,168 and 5,618,487 to Harbor Branch Oceanographic Institute or by chemical synthesis as described, for example, in GB 2280677, WO 98/24429 and U.S. Pat. Nos. 5,789,605 and 6,031,133, which are here incorporated by reference. Etoposide can be administered, e.g., in the form as it is marketed, e.g. under the trademark ETOPOPHOS™. Teniposide can be administered, e.g., in the form as it is marketed, e.g. under the trademark VM 26-BRISTOL™.

Discodermolide, as well as its analogs and derivatives, are especially useful MIA compounds. Discodermolide and its preparation are known in the art. The preparation of analogs and derivatives has also been reported in the literature.

Epothilones that can be used in the present invention are described by formula (I), 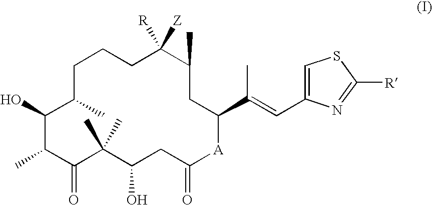
wherein A represents O or NR_{N}, wherein R_{N} is hydrogen or lower alkyl, R is hydrogen or lower alkyl, R′ is methyl, methoxy, ethoxy, amino, methylamino, dimethylamino or methylthio, and Z is O or a bond.

Unless stated otherwise, in the present disclosure organic radicals and compounds designated “lower” contain not more than 7, preferably not more than 4, carbon atoms.

A compound of formula I wherein A represents O, R is hydrogen, R′ is methyl and Z is O is known as epothilone A; a compound of formula I wherein A represents O, R is methyl, R′ is methyl and Z is 0 is known as epothilone B; a compound of formula I wherein A represents O, R is hydrogen, R′ is methyl and Z is a bond is known as epothilone C; a compound of formula I wherein A represents O, R is methyl, R′ is methyl and Z is a bond is known as epothilone D.

Epothilone derivatives of formula I wherein A represents O or NR_{N}, wherein R_{N} is hydrogen or lower alkyl, R is hydrogen or lower alkyl, R′ is methyl and Z is O or a bond, and methods for the preparation of such epothilone derivatives are in particular generically and specifically disclosed in the patents and patent applications WO 93/10121, U.S. Pat. No. 6,194,181, WO 98/25929, WO 98/08849, WO 99/43653, WO 98/22461 and WO 00/31247 in each case in particular in the compound claims and the final products of the working examples, the subject-matter of which is hereby incorporated into the present application by reference to this publications. Comprised are likewise the corresponding stereoisomers as well as the corresponding crystal modifications, e.g. solvates and polymorphs, which are disclosed therein.

Epothilone derivatives of formula I wherein A represents O or NR_{N}, wherein R_{N} is hydrogen or lower alkyl, R is hydrogen or lower alkyl, R′ is methoxy, ethoxy, amino, methylamino, dimethylamino or methylthio, and Z is O or a bond, and methods for the preparation of such epothilone derivatives are in particular generically and specifically disclosed in the patent application WO99/67252, which is hereby incorporated by reference into the present application. Comprised are likewise the corresponding stereoisomers as well as the corresponding crystal modifications, e.g. solvates and polymorphs, which are disclosed therein.

The transformation of epothilone B to the corresponding lactam is disclosed in Scheme 21 (page 31, 32) and Example 3 of WO 99/02514 (pages 48-50). The transformation of a compound of formula I which is different from epothilone B into the corresponding lactam can be accomplished analogously. Corresponding epothilone derivatives of formula I wherein R_{N} is lower alkyl can be prepared by methods known in the art such as a reductive alkylation reaction starting from the epothilone derivative wherein R_{N} is hydrogen.

Preferably, the invention relates to a method of treating for preventing and treating intimal thickening or restenosis that occurs after injury, e.g. vascular injury, including e.g. surgical injury, e.g. revascularization-induced injury, e.g. also in heart or other grafts, comprising administering a therapeutically effective amount of a compound of formula I wherein A represents O or NR_{N}, wherein R_{N} is hydrogen or lower alkyl, R is hydrogen or lower alkyl, R′ is methyl, methoxy, ethoxy, amino, methylamino, dimethylamino or methylthio, and Z is O or a bond, to a warm-blooded animal, preferably a human, in need thereof.

According to the invention, the MIA may be applied as the sole active ingredient or in conjunction with an immunosuppressive agent, e.g. a calcineurin inhibitor, e.g. a cyclosporin, for example cyclosporin A, or FK506, an EDG-Receptor agonist, e.g. FTY720, an antiinflammatory agent, e.g. a steroid, e.g. a corticosteroid, e.g. dexamethasone or prednisone, a NSAID, e.g. a cyclooxygenase inhibitor, e.g. a COX-2 inhibitor, e.g. celecoxib, rofecoxib, etoricoxib or valdecoxib, or an ascomycin, e.g. ASM981, an anti-thrombotic or anticoagulant agent, e.g. heparin, a IIb/IIIa inhibitor, etc. an antiproliferative agent, a tyrosine kinase inhibitor which is not an inhibitor of VEGF, e.g. staurosporin and related small molecules, e.g. UCN-01, BAY 43-9006, Bryostatin 1, Perifosine, Limofosine, midostaurin, RO318220, RO320432, GO 6976, Isis 3521, LY333531, LY379196, SU5416, SU6668, AG1296 etc., a compound or antibody which inhibits the PDGF receptor tyrosine kinase or a compound which binds to PDGF or reduces expression of the PDGF receptor e.g. STI571, CT52923, RP-1776, GFB-111, pyrrolo[3,4-c]-beta-carboline-diones, etc., a compound or antibody which inhibits the EGF receptor tyrosine kinase or a compound which binds to EGF or reduces expression of the EGF receptor e.g. the compounds disclosed in WO97/02266, e.g. the compound of example 39, retinoic acid, ZD1839 (Iressa), alpha-, gamma- or delta-tocopherol or alpha-, gamma- or delta-tocotrienol, or compounds affecting GRB2, IMC-C225, a statin, e.g. having HMG-CoA reductase inhibition activity, e.g. fluvastatin, lovastatin, simvastatin, pravastatin, atorvastatin, cerivastatin, pitavastatin, rosuvastatin or nivastatin, a compound, protein, growth factor or compound stimulating growth factor production that will enhance endothelial regrowth of the luminal endothelium, e.g. FGF, IGF, a matrix metalloproteinase inhibitor, e.g. batimistat, marimistat, trocade, CGS 27023, RS 130830 or AG3340, a modulator (i.e. antagonists or agonists) of kinases, e.g. JNK, ERK1/2, MAPK or STAT, or a compound stimulating the release of (NO) or a NO donor, e.g. diazeniumdiolates, S-nitrosothiols, mesoionic oxatriazoles, a combination of isosorbide.

The present invention also provides the local administration or delivery of an MIA in conjunction with a calcineurin inhibitor, e.g. as disclosed above, an EDG-Receptor agonist, e.g. as disclosed above, a compound or antibody which inhibits the PDGF receptor tyrosine kinase or a compound which binds to PDGF or reduces expression of the PDGF receptor, e.g. as disclosed above, a compound or antibody which inhibits the EGF receptor tyrosine kinase or a compound which binds to EGF or reduces expression of the EGF receptor, e.g. as disclosed above, a statin, e.g. as disclosed above, a compound, protein, growth factor or compound stimulating growth factor production that will enhance endothelial regrowth of the luminal endothelium, e.g. as disclosed above, a matrix metalloproteinase inhibitor, e.g. as disclosed above, an inhibitor of a modulator (i.e. antagonists or agonists) of kinases, e.g. as disclosed above, or a compound stimulating the release of (NO) or a NO donor, e.g. as disclosed above.

In accordance with the particular findings of the present invention, there is provided:

1. A method for preventing or treating smooth muscle cell proliferation and migration in hollow tubes, or increased cell proliferation or decreased apoptosis or increased matrix deposition in a mammal in need thereof, comprising local administration of a therapeutically effective amount of an MIA, optionally in conjunction with one or more other active ingredients, e.g. as disclosed above.

2. A method for the treatment of intimal thickening in vessel walls comprising the controlled delivery from any catheter-based device or intraluminal medical device of a therapeutically effective amount of an MIA, optionally in conjunction with one or more other active ingredients, e.g. as disclosed above. Preferably the disease to be treated is stenosis, restenosis, e.g. following revascularization or neovascularization, and/or inflammation and/or thrombosis.

3. A drug delivery device or system comprising a) a medical device adapted for local application or administration in hollow tubes, e.g. a catheter-based delivery device or intraluminal medical device, and b) a therapeutic dosage of an MIA, optionally in conjunction with a therapeutic dosage of one or more other active ingredients, e.g. as disclosed above, each being releasably affixed to the catheter-based delivery device or medical device.
Such a local delivery device or system can be used to reduce stenosis or restenosis as an adjunct to revascularization, bypass or grafting procedures performed in any vascular location including coronary arteries, carotid arteries, renal arteries, peripheral arteries, cerebral arteries or any other arterial or venous location, to reduce anastomic stenosis such as in the case of arterial-venous dialysis access with or without polytetrafluoroethylene grafting and with or without stenting, or in in conjunction with any other heart or transplantation procedures, or congenital vascular interventions.

An MIA will be referred to hereinafter as “drug”. The other active ingredients which may be used in conjunction with the MIA, e.g. as disclosed above, will be referred to hereinafter collectively as “adjunct”. Drug(s) shall mean drug or drug plus adjunct.

The local administration preferably takes place at or near the vascular lesions sites.

The administration may be by one or more of the following routes: via catheter or other intravascular delivery system, intranasally, intrabronchially, interperitoneally or eosophagal. Hollow tubes include circulatory system vessels such as blood vessels (arteries or veins), tissue lumen, lymphatic pathways, digestive tract including alimentary canal, respiratory tract, excretory system tubes, reproductive system tubes and ducts, body cavity tubes, etc. Local administration or application of the drug(s) affords concentrated delivery of said drug(s), achieving tissue levels in target tissues not otherwise obtainable through other administration route.

Means for local drug(s) delivery to hollow tubes can be by physical delivery of the drug(s) either internally or externally to the hollow tube. Local drug(s) delivery includes catheter delivery systems, local injection devices or systems or indwelling devices. Such devices or systems would include, but not be limited to, stents, coated stents, endolumenal sleeves, stent-grafts, liposomes, controlled release matrices, polymeric endoluminal paving, or other endovascular devices, embolic delivery particles, cell targeting such as affinity based delivery, internal patches around the hollow tube, external patches around the hollow tube, hollow tube cuff, external paving, external stent sleeves, and the like. See, Eccleston et al. (1995) Interventional Cardiology Monitor 1:33-40-41 and Slepian, N.J. (1996) Intervente. Cardiol. 1:103-116, or Regar E, Sianos G, Serruys P W. Stent development and local drug delivery. Br Med Bull 2001, 59:227-48 which disclosures are herein incorporated by reference.

By “biocompatible” is meant a material which elicits no or minimal negative tissue reaction including e.g. thrombus formation and/or inflammation.

Delivery or application of the drug(s) can occur using stents or sleeves or sheathes. An intraluminal stent composed of or coated with a polymer or other biocompatible materials, e.g.: porous ceramic, e.g. nanoporous ceramic, into which the drug(s) has been impregnated or incorporated can be used. Such stents can be biodegradable or can be made of metal or alloy, e.g. Ni and Ti, or another stable substance when intented for permanent use. The drug(s) may also be entrapped into the metal of the stent or graft body which has been modified to contain micropores or channels. Also lumenal and/or ablumenal coating or external sleeve made of polymer or other biocompatible materials, e.g. as disclosed above, that contain the drug(s) can also be used for local delivery.

Stents are commonly used as a tubular structure left inside the lumen of a duct to relieve an obstruction. They may be inserted into the duct lumen in a non-expanded form and are then expanded autonomously (self-expanding stents) or with the aid of a second device in situ, e.g. a catheter-mounted angioplasty balloon which is inflated within the stenosed vessel or body passageway in order to shear and disrupt the obstructions associated with the wall components of the vessel and to obtain an enlarged lumen.

For example, the drug(s) may be incorporated into or affixed to the stent in a number of ways and utilizing any biocompatible materials; it may be incorporated into e.g. a polymer or a polymeric matrix and sprayed onto the outer surface of the stent. A mixture of the drug(s) and the polymeric material may be prepared in a solvent or a mixture of solvents and applied to the surfaces of the stents also by dip-coating, brush coating and/or dip/spin coating, the solvent (s) being allowed to evaporate to leave a film with entrapped drug(s). In the case of stents where the drug(s) is delivered from micropores, struts or channels, a solution of a polymer may additionally be applied as an outlayer to control the drug(s) release; alternatively, the drug may be comprised in the micropores, struts or channels and the adjunct may be incorporated in the outlayer, or vice versa. The drug may also be affixed in an inner layer of the stent and the adjunct in an outer layer, or vice versa. The drug(s) may also be attached by a covalent bond, e.g. esters, amides or anhydrides, to the stent surface, involving chemical derivatization. The drug(s) may also be incorporated into a biocompatible porous ceramic coating, e.g. a nanoporous ceramic coating.

Examples of polymeric materials include biocompatible degradable materials, e.g. lactone-based polyesters or copolyesters, e.g. polylactide; polylactide-glycolide; polycaprolactone-glycolide; polyorthoesters; polyanhydrides; polyaminoacids; polysaccharides; polyphosphazenes; poly(ether-ester) copolymers, e.g. PEO-PLLA, or mixtures thereof; and biocompatible non-degrading materials, e.g. polydimethylsiloxane; poly(ethylene-vinylacetate); acrylate based polymers or coplymers, e.g. polybutylmethacrylate, poly(hydroxyethyl methylmethacrylate); polyvinyl pyrrolidinone; fluorinated polymers such as polytetrafluoethylene; cellulose esters.

When a polymeric matrix is used, it may comprise 2 layers, e.g. a base layer in which the drug(s) is/are incorporated, e.g. ethylene-co-vinylacetate and polybutylmethacrylate, and a top coat, e.g. polybutylmethacrylate, which is drug(s)-free and acts as a diffusion-control of the drug(s). Alternatively, the drug may be comprised in the base layer and the adjunct may be incorporated in the outlayer, or vice versa. Total thickness of the polymeric matrix may be from about 1 to 20μ or greater.

According to the method of the invention or in the device or system of the invention, the drug(s) may elute passively, actively or under activation, e.g. light-activation.

The drug(s) elutes from the polymeric material or the stent over time and enters the surrounding tissue, e.g. up to ca. 1 month to 1 year. The local delivery according to the present invention allows for high concentration of the drug(s) at the disease site with low concentration of circulating compound. The amount of drug(s) used for local delivery applications will vary depending on the compounds used, the condition to be treated and the desired effect. For purposes of the invention, a therapeutically effective amount will be administered. By therapeutically effective amount is intended an amount sufficient to inhibit cellular proliferation and resulting in the prevention and treatment of the disease state. Specifically, for the prevention or treatment of restenosis e.g. after revascularization, or antitumor treatment, local delivery may require less compound than systemic administration.

Utility of the drug(s) may be demonstrated in animal test methods as well as in clinic, for example in accordance with the methods hereinafter described. The following examples are illustrative of the invention without limitating it.

A1. Inhibition of Late Neointimal Lesion Formation in the 28 Day Rat Carotid Artery Balloon Injury Model

Numerous compounds have been shown to inhibit intimal lesion formation at 2 weeks in the rat ballooned carotid model, while only few compounds prove effective at 4 weeks. Compounds of formula I are tested in the following rat model.

Rats are dosed orally with placebo or a MIA, e.g. a compound of formula I, e.g. epothilone B. Daily dosing starts 3 days prior to surgery and continues for 31 days. Rat carotid arteries are balloon injured using a method described by Clowes et al. Lab. Invest. 1983; 49; 208-215. Quantitation of vascular inflammatory cell number is performed using cell flow cytometry as described by Hay C. et al., Arterioscler. Thromb. Vasc. Biol. 21 (2001) 1948-1954. In studies determining lesion size, BrDU is administered for 24 hours prior to sacrifice. Sacrifice is performed at 1, 9 or 21 days post-balloon injury. Carotid arteries are removed and processed for histologic and morphometric evaluation.

In this assay, the ability of a compound of formula I, e.g. epothilone B can be demonstrated to significantly reduce CD45-positive leukocyte infiltration into the vessel wall and adventitia at 1 day and to significantly reduce neointimal lesion formation following balloon injury at 9 and 12 days. Furthermore, when a MIA, e.g. a compound of formula I, e.g. epothilone B is administered locally to the adventitia adjacent to the ballooned carotid (via a cather implanted into the adventitia that is connected to an Alzet minipump containing a MIA, e.g. a compound of formula I, e.g. epothilone B suspended in vehicle), there is potent inhibition of infiltration of CD45+ leukocytes at day 1 and both early (9 days post-ballooning) and late (21-31 days post-ballooning) neointimal lesions, as well as potent inhibition of constrictive remodeling.

A.2 Inhibition of Restenosis at 28 Days in the Rabbit Iliac Stent Model

A combined angioplasty and stenting procedure is performed in New Zealand White rabbit iliac arteries. Iliac artery balloon injury is performed by inflating a 3.0×9.0 mm angioplasty balloon in the mid-portion of the artery followed by “pull-back” of the catheter for 1 balloon length. Balloon injury is repeated 2 times, and a 3.0×12 mm stent is deployed at 6 atm for 30 seconds in the iliac artery. Balloon injury and stent placement is then performed on the contralateral iliac artery in the same manner. A post-stent deployment angiogram is performed. All animals receive oral aspirin 40 mg/day daily as anti-platelet therapy and are fed standard low-cholesterol rabbit chow. Twenty-eight days after stenting, animals are anesthetized and euthanized and the arterial tree is perfused at 100 mmHg with lactated Ringer's for several minutes, then perfused with 10% formalin at 100 mmHg for 15 minutes. The vascular section between the distal aorta and the proximal femoral arteries is excised and cleaned of periadventitial tissue. The stented section of artery is embedded in plastic and sections are taken from the proximal, middle, and distal portions of each stent. All sections are stained with hematoxylin-eosin and Movat pentachrome stains. Computerized planimetry is performed to determine the area of the internal elastic lamina (IEL), external elastic lamina (EEL) and lumen. The neointima and neointimal thickness is measured both at and between the stent struts. The vessel area is measured as the area within the EEL. Data are expressed as mean±SEM. Statistical analysis of the histologic data is accomplished using analysis of variance (ANOVA) due to the fact that two stented arteries are measured per animal with a mean generated per animal. A P<0.05 is considered statistically significant.

An MIA, e.g. a compound of formula 1, e.g. epothilone B is administered orally by gavage at an initial dose one day prior to stenting, then dosed at 50% of the initial dose from the day of stenting until day 27 post-stenting. In this model a marked reduction in the extent of restenotic lesion formation in the presence of an MIA, e.g. a compound of formula I, e.g. epothilone B can be shown, whereas there is extensive neointimal formation in placebo-treated animals at 28 days, with the lesions consisting of abundant smooth muscle cells in proteoglycan/collagen matrix and apparent full endothelial healing. In addition, lesion formation in the portions of artery immediately proximal and immediately distal to the stent is also inhibited in the animals treated with an MIA, e.g. a compound of formula I, e.g. epothilone B compared to those treated with placebo. Furthermore, the number of inflammatory cells, especially those in the area surrounding the stent struts, is significantly reduced in MIA, e.g. a compound of formula I, e.g. epothilone B samples compared to those treated with placebo.

A.3 Manufacture of a Stent

A stent (e.g. a Multi-Link Vision stent, Guidant Corp.; or a DRIVER stent, Medtronic Corp.) is weighed and then mounted on a rotating or other support for coating with a polymer or other synthetic or biologic carrier used as a drug reservoir. In an example of an application of one such carrier, while the stent is rotating, a 100 μl aliquot of a solution of polylactide glycolide, 0.75 mg/ml of (+)-discodermolide and 0.0015 mg/ml 2,6-di-tert-butyl-4-methylphenol dissolved in a 50:50 mixture of methanol and tetrahydrofuran, is coated onto it. The coated stent is removed from the support and allowed to air-dry. After a final weighing the amount of coating on the stent is determined.

A.4 (+)-Discodermolide Release from Polymer Coatings in Aqueous Solution

Four 2 cm pieces of coated stents as described above are placed into 100 mL of phosphate buffer solution (PBS) having a pH of 7.4. Another 4 pieces from each series are placed into 100 mL of polyethylene glycol (PEG)/water solution (40/60 v/v, MW of PEG=400). The stent pieces are incubated at 37° C. in a shaker. The buffer and PEG solutions are changed daily and different assays are performed on the solution to determine the released (+)-discodermolide concentrations. By such method a stable (+)-discodermolide release from coated stents can be shown. The term “stable (+)-discodermolide release” means that less than 10% of variation of the drug release rate is observed.

A.5 (+)-Discodermolide Release from Polymer Coatings in Plasma

Release of (+)-discodermolide in plasma can also be studied. 1 cm pieces of a coated stent are put into 1 mL of citrated human plasma (from Helena Labs.) in lyophilized form and reconstituted by adding 1 mL of sterile deionized water. Three sets of stent plasma solutions are incubated at 37° C. and the plasma is changed daily. Different assays are performed on the solution to determine the released (+)-discodermolide concentrations. By such method a stable (+)-discodermolide release from coated stents in plasma can be demonstrated.

A.6 Drug Stability in Pharmaceutically Acceptable Polymers at Body Temperature

PDGF-stimulated receptor tyrosine kinase assay can be performed on the last piece of each sample to determine the MIA, e.g. a compound of formula I, e.g. epothilone B activity. A similar test can be performed with free MIA, e.g. a compound of formula I, e.g. epothilone B. The inhibition of PDGF-stimulated receptor tyrosine kinase activity in vitro can be measured in PDGF receptor immunocomplexes of BALB/c 3T3 cells, analogously to the method described by E. Andrejauskas-Buchdunger and U. Regenass in Cancer Research 52 (1992) 5353-5358. By such approach the stability of free MIA, e.g. a compound of formula I, e.g. epothilone B and MIA, e.g. a compound of formula I, e.g. epothilone B in polymer coatings can be compared.

In A1 to A6 pimecrolimus may be replaced with Epothilone B, Discodermolide or with similar results.

Clinical Trial

The favorable effects of the anti inflammatory ascomycin derivative pimecrolimus used according to the invention can furthermore be demonstrated in a randomized, double blind multi-center trial for revascularization of single, primary lesions in native coronary arteries, e.g. along the following lines:

The primary endpoint is in-stent late luminal loss (difference between the minimal luminal diameter immediately after the procedure and the diameter at six months). Secondary endpoints include the percentage of in-segment stenosis (luminal diameter of stented portion plus the 5 mm proximal to and distal from the stented portion of the vessel), and the rate of repeat revascularization needed at the site of target vessel stenting. After six months, the degree of neointimal proliferation, manifested as the mean late luminal loss in the group treated with a coated stent comprising pimecrolimus versus the placebo group treated with a non-coated stent is determined, e.g. by means of a virtual, conventional catheter-based coronary angiography, and/or by means of intracoronary ultrasound.

1. A method for preventing or treating smooth muscle cell proliferation and migration in hollow tubes or increased cell proliferation or decreased apoptosis or increased matrix deposition in a mammal in need thereof, comprising local administration of a therapeutically effective amount of a microtubule interfereing agents (MIA).

2. A method for the treatment of intimal thickening in vessel walls comprising the controlled delivery from a catheter-based device or an intraluminal medical device of a therapeutically effective amount of a microtubule interfereing agents (MIA).

3. The method according to claim 1 wherein the MIA is an epothilone of formula (I), 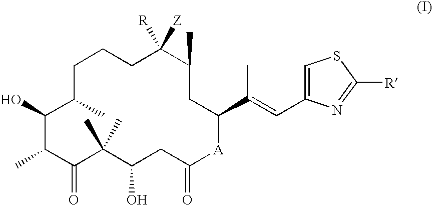
wherein A represents O or NR_{N}, wherein R_{N} is hydrogen or lower alkyl, R is hydrogen or lower alkyl, R′ is methyl, methoxy, ethoxy, amino, methylamino, dimethylamino or methylthio, and Z is O or a bond.

4. The method according to claim 1 wherein the MIA is (+)-discodermolide.

5. The method according to claim 1 wherein the administration or delivery is intravascular, intranasal, intrabronchial, interperitoneal or eosophagal.

6. The method according to claim 1 wherein the administration or delivery is made using a catheter delivery system, a local injection device, an indwelling device, a stent, a coated stent, a sleeve, a stem-graft, polymeric endoluminal paving or a controlled release matrix.

7. The method according to claim 1 wherein the microtubule interfereing agents (MIA) is administered from a stent or from a coating applied to a stent.

8. A method according to claim 2 wherein the microtubule interfereing agents (MIA) is delivered from a stent or from a coating applied to a stent.

9. A method according to claim 1 for the treatment of stenosis, restenosis or inflammation.

10. A method according to claim 2 for the treatment of stenosis, restenosis or inflammation.

11. A drug delivery device or system comprising a) a medical device adapted for local application or administration in hollow tubes, e.g. a catheter-based delivery device or an intraluminal medical device, and b) a therapeutic dosage of a microtubule interfereing agents (MIA) being releasably affixed to the medical device.

12. The device according to claim 11 comprising (+)-discodermolide.

13. The device according to claim 11 comprising epothilone B or epothilone D.

14. The device according to claim 11 comprising [1S-[1R, 3R(E), 7R, 10S, 11R, 12R, 16S]]-7,11-dihydroxy-8,8,10,12,16-pentamethyl-3-[1-methyl-2-(2-methyl-4-thiazolyl)ethenyl]-4-aza-17-bicyclo[14.1.0]-heptadecane-5,9-dione.

15. The device according to claim 11 which is a catheter delivery system, a local injection device, an indwelling device, a stent, a stem-graft or a sleeve.

16. A device according to claim 11 which is a coated stent.
